(19) United States
(12) Patent Application Publication
(10) Pub. No.: US 2005/0106699 A1
(43) Pub. Date: May 19, 2005

(54) Process for xylanase production

(76) Inventors: Vanga Siva Reddy, New Delhi (IN); Sadhu Leelavathi, New Delhi (IN); Naveen Gupta, Chandigarh (IN); Sankar Maiti, Chandigarh (IN); Amit Ghosh, Chandigarh (IN)

Correspondence Address: LADAS & PARRY, 26 WEST 61ST STREET, NEW YORK, NY, 10023, US

(21) Appl. No.: 10/485,347

(22) PCT Filed: Aug. 2, 2002
(86) PCT No.: PCT/EP02/08655

(30) Foreign Application Priority Data
Aug. 2, 2001 (GB) 0118935.6

(51) Int. Cl. C12N 9/24; A01H 1/00; C12N 15/87; C12N 5/04

(52) U.S. Cl. 435/200; 435/419; 800/284

(57) ABSTRACT

The invention provides a process of obtaining a xylanase, said process comprising: providing a protein-containing extract of a transplastomic plant tissue comprising plastids transformed with a polynucleotide encoding said xylanase, said extract having been subjected to heat treatment that has denatured at least some of the protein content of said tissue but under which the xylanase has remained stable; and recovering said xylanase from said extract.

### FIELD OF THE INVENTION

The present invention is in the field of plant biotechnology. It relates more particularly to the transformation of a plastid genome with a polynucleotide encoding xylanase, and to the production of xylanase thereby.

### BACKGROUND OF THE INVENTION

Plastids

Plastids are organelles found in plant cells. Various plastids exist and are derived from undifferentiated proplastids. Differentiated plastids include amyloplasts, chromoplasts, chloroplasts, etioplasts and leucoplasts. Chloroplasts are the most common plastids, and are the site of photosynthesis. Each photosynthetic cell contains multiple chloroplasts, typically from 50 to 100. Chloroplasts have their own genome, the plastome, which exists in addition to the main cellular (nuclear) genome, and transcription and translation systems. The latter resemble prokaryotic transcription and translation systems. Each chloroplast contains multiple genome copies, typically from 50 to 100. A plastid genome, referred to as a plastome, comprises a double stranded circular DNA molecule.

Nuclear Transformation

Typically, transgene expression in plants is achieved by the integration of a transgene construct into nuclear DNA. In the majority of transformation experiments using Agrobacterium and/or gene guns, the number of copies of the transgene integrated into the transformed plant nuclear genome is typically low, and expression levels achieved are also low. Expression may also be affected by other factors, such as the site of integration. This means that the levels of expression achieved by independently derived nuclear transformed plants harbouring the same transgene can also be highly variable.

Plant zygotes contain nuclear DNA derived from both the female (ova) and male (pollen) gametes, both of which contribute to the characteristics of the mature plant. Therefore, nuclear-encoded transgenes can be spread in the ecosystem by the dispersal of pollen, which contains the male gametes, from plants containing a nuclear transgene and subsequent fertilisation of wild type plants. The dispersal of pollen derived from a nuclear transformed plant, therefore, provides a potential vehicle for the unwanted “lateral” transmission of transgenes into other species. There is considerable concern about tis, especially over the possible transmission of herbicide/insecticide/disease resistance traits from transgenic crops to weedy relatives growing around the crop fields, leading to the possibility of resistant weeds (so-called “super-weeds”) which are hard to eliminate because of their resistant traits.

Chloroplast Transformation

Many of the disadvantages of nuclear transformation can be avoided by targeting transgene integration to the plastome. A transformed plastome is referred to as a transplastome. Due to the existence of multiple plastome copies within each chloroplast, the copy number of an integrated transgene is high. This leads to a level of expression of a transplastomic gene that is typically higher than for an equivalent transgene integrated into nuclear DNA. Such plants are referred to as transplastomic plants. Plastids are maternally inherited. That is, zygotes derive plastids from the cytoplasm inherited from the female gamete, whereas pollen does not contribute plastids to the zygote. Pollen derived from transplastomic plants does not, therefore, contain the transgene and so transgene transmission to other species is not possible. This is particularly beneficial in view of public fears related to the spread of transgenes and their potential impact on the ecosystem.

Foreign DNA has previously been introduced into chloroplasts using a biolistic method (Boynton et al, 1988; Svab et al, 1990; Svab and Maliga, 1993; U.S. Pat. No. 5,451,513; U.S. Pat. No. 5,545,817; U.S. Pat. No. 5,545,818; U.S. Pat. No. 5,576,198; U.S. Pat. No. 5,866,421) and a PEG-based procedure (Golds et al, 1993). Typically, the transgene in a chloroplast transformation vector is flanked by DNA regions homologous to regions of the plastome. These flanking regions enable the site-specific integration of the transgene construct into plastome by the process of homologous recombination, a process which naturally occurs in plastids. Therefore, the site of transgene integration is more assured in chloroplast-based techniques relying on homologous recombination than in nuclear-based processes. Therefore, more uniform transgene expression results between independently derived transplastomic plants than between independently derived nuclear transformed ones. Improved techniques for high, uniform, reliable transplastomic expression are provided in PCT/EP00/12446, published as WO01/42441.

Hemicellulose and Xylanase

Hemicellulose is the second most abundant renewal polysaccharide in nature after cellulose. β-1,4-xylan is a major component of hemicellulose and has a backbone of β-1,4-linked D-xylopyranoside residues substituted with acetyl, arabinosyl and uronyl side chains. Complete digestion of xylan requires the action of several hydrolytic enzymes, the most important among which is endo-1,4-xylanse (EC 3.2.1.8). Xylanases have been detected in a number of microorganisms and thermostable xylanases are of special interest for their potential use in: (1) paper industry for the production of pulp with improved qualities, (2) baking, brewing and feed industry for the improvement of product quality, (3) conversation of xylan to monosaccharides that can be further converted into ethanol, (4) the preparation of complex polysaccharide diet for monogastric animals and, (5) processing of plant fibers (e.g. flax and hemp) by selectively removing xylan components (Herbers et al, 1995; Liu et al, 1997). Despite these important applications, currently xylanases are not being used routinely by the industry mainly because of the high costs involved in their production (Liu et al, 1997).

WO95/12668 reports the cloning and expression in bacteria of the xylanase XynA from the fungus Thermonospora fusca. Cellulolytic enzymes have been expressed in filamentous fungi (WO97/27306).

Production of cellulolytic enzymes in plants had been a major challenge as these enzymes can potentially degrade the cell wall components of the very cell that is expressing these enzymes, affecting the normal growth and development of the transgenic plants. Xylanase genes have been expressed in plants by targeting the recombinant enzyme to accumulate in the intercellular space (Herbers et al, 1995), in the oil body membrane in seeds (Liu et al, 1997) and by secreting the enzyme through roots into a hydroponic culture medium (Borisijuk et al, 1999). In all these cases, the xylanase gene was introduced into the nuclear genome of the target plant. The expression levels were low. Nuclear transformation of B. napus with xylanase XynC from the fungus Neocallimastix patricarum is also reported in U.S. Pat. No. 6,137,032.

### SUMMARY OF THE INVENTION

Plastids have been transformed with cellulase genes (WO98/11235, U.S. Pat. No. 6,013,860). Plastid transformation with xylanase genes has not been previously been reported. We have transformed the xynA gene coding for an alkali and thermostable xylanase from a mesophilic obligate alkalophilic Bacillus sp. NG-27 into chloroplast genome of tobacco plants. We report here the successful high level expression and purification of this industrially important enzyme and thus provide its significant benefits related to technical industry, agriculture and the environment.

For the first time, we have shown that chloroplasts can overexpress and contain a cellulolytic alkali and thermostable xylanase in large amounts without any harmful effects on plant growth for generations. The expression levels of xylanase were found to be very high, reaching up to 6% of the total soluble protein. The recombinant protein was purified to more than 95% homogeneity by simply heating the crude leaf extract to 60° C. followed by ammonium sulfate precipitation, and without any involvement of conventional chromatography techniques. This is advantageous because plant bioreactor systems have a much higher ratio of biomass to recombinant proteins than yeast or E. coli-based expression systems (˜10,000:1 for plants, ˜100:1 for microbial systems). Thus, simple, effective, large-scale purification techniques are particularly import in plant-based systems. 95% purity may be sufficient for direct use in the pulp industry and the enzyme was purified further for use in the animal feed and bakery industries via conventional chromatography techniques.

Surprisingly, the enzyme was active even in leaves that had undergone senescence and that had been dried at 42° C. or sun-dried, with a recovery of 85% activity. This finding is of utmost importance to the farmer in judging the time to harvest the leaf material and store them until a desired price is realised. It also offers enormous flexibility for transportation, storage and in the initial stages of extraction.

The chloroplast-expressed xylanase retained its substrate specificity, pH and temperature optima. Most importantly, the transgenic plants were indistinguishable from the control untransformed plants in their morphology, growth and development and in seed setting.

These results open up an excellent and simple system for the cost-effective production of xylanases in large quantities for various industrial applications. This has not been possible through any other transformation system in plants.

Accordingly, the present invention provides:
a process of obtaining a xylanase, said process comprising:
   providing a protein-containing extract of a transplastomic plant tissue comprising plastids transformed with a polynucleotide encoding said xylanase, said extract having been subjected to heat treatment that has denatured at least some of the protein content of said tissue but under which the xylanase has remained stable; and
   recovering said xylanase from said extract.

The invention also provides:
a transplastome transformed with a polynucleotide encoding a xylanase.

The invention also provides:
a transplastomic or homotransplastomic plastid comprising such a transplastome.

The invention also provides:
a transplastomic or homotransplastomic cell comprising such a plastid, or a transplastomic or homotransplastomic plant, plant seed, or plant tissue comprising said cell.

The invention also provides:
a process of obtaining a xylanase comprising expressing said xylanase in such a cell, plant, seed or tissue.

The invention also provides:
a xylanase obtained by a process of the invention.

The invention also provides:
use of a xylanase obtained a process of the invention in the manufacture of paper; for improvement of product quality in baked or brewed products or feed; in the conversion of xylan to polysaccharides, optionally for further conversion to ethanol; in the preparation of complex polysaccharide diets for monogastric animals; or in the processing of plant fibres by selective removal of xylan components.

### BRIEF DESCRIPTION OF THE DRAWINGS

FIG. 1: Restriction map of vector p326xynA, partial chloroplast DNA of tobacco (cpDNA) and the transformed tobacco plant (Nt. 326xynA-1) plastid DNA

Lines indicate the size of DNA fragments after the restriction digestion with various enzymes. Direction (dashed arrow) and size of the xynA transcript are also indicated. A possible mechanism for site-specific integration of aadA and xynA through two homologous recombinations (crossed lines) is also shown. XhoI (Xh), ClaI (C), BamHI (B), XbaI (X), PstI (P).

FIG. 2: Zymography to detect the activity of xylanase in chloroplast transformed plant leaf

Top panel: leaves from wild type (left) and chloroplast transformed (right) plants were pressed against fine (0-grade) sand paper and placed on agar gel containing 1% xylan. After incubation at 70° C. for 1 hour the zymogram was developed with Congo Red (bottom panel). Note the presence of xylanase activity throughout the surface area covered by the transformed plant leaf.

FIG. 3:. SDS-PAGE analysis of protein samples from various stages of purification

For a direct comparison, protein samples were processed from a wild type (WT), untransformed plant and a chloroplast plant (PT). Arrow indicates the expected size band (42 kDa) for the xylanase.

FIG. 4: SDS-PAGE analysis for the detection of xylanase and its activity in the Sun dried leaves (A) and the leaves undergoing senescence (B)

Crude extra after heat treatment at 60° C. for 30 minutes were separated on SDS-PAGE and assayed for the xylanase activity (bottom panel). Note the presence of a band corresponding to 42 kDa in the transformed (PT) and absent in the wild type (WT) plant extracts.

FIG. 5: Zymography for assessing the temperature requirement for the optimum activity of xylanase produced in chloroplasts of tobacco

Arrow indicates activity zone of the xylanase and the band corresponds with the expected molecular size for the XynA.

FIG. 6: Substrate specificity of chloroplast expressed enzyme

Specificity was determined using oat spelt xylan. On a paper chromatograp, it shows that the major hydrolysis products of xylan were xylobiose and zylose. Lane (1) maltose, (2) xylose, (3) xylobiose, xylan treated with plant produced xylanase (4) and bacterial produced xylanase (5).

FIG. 7: Purification of xylanase

Leaf extracts after heat treatment at 70° C. for 30 minutes were loaded on to Q-sepharose column and eluted with a NaCl gradient (0 M to 1.0 M). Protein elution profile and xylanase activity (A). Identification of fractions containing xylanase activity (B). Note the change of colour from light yellow to brown. The protein profile of fractions containing xylanase activity on SDS-PAGE. Note the presence of a single band corresponding to 42 kDa in the active fractions. M, molecular marker, C, control fraction without the enzyme. Activity of xylanase was measured at 550 nm as described in the materials and methods.

### DETAILED DESCRIPTION OF Two INVENTION

Plastids

Plastids suitable for use in this invention may be derived from any organism that has plastids. They may be derived from any cell type and may be of any differentiated or undifferentiated state. Such states include undifferentiated proplastid, amyloplast, chromoplast, chloroplast, etioplast, leucoplast. Preferably, the plastid will be a chloroplast.

Plastids comprise their own genome, herein referred to as a plastome. Typically individual plastids comprise multiple plastomes, more typically from 5 to 500, most typically from 50 to 100. Herein, a recipient plastome is one that may be transformed with a xylanase-encoding polynucleotide of the invention, as described below.

Herein, a recipient plastome transformed with a xylanase-encoding polynucleotide according to the invention is referred to as a transplastome. Plastids comprising a transplastome are referred to as transplastomic. Plastids wherein all plastomes are identical, or substantially identical, transplastomes are referred to as homotransplastomic. In this context, the plastomes of plastids are substantially identical if they all comprise the coding region of the transforming polynucleotide of the invention, and preferably any associated regulatory sequences, or at least enough of the coding regulatory sequences to secure expression of the coding sequence. Cells containing plastids are homotransplastomic if all the plastids in the cell are homotransplastomic. Plants, plant parts and seeds are homotransplastomic if all of their cells are homotransplastomic.

Plastomes and Plants of the Invention

The invention maybe applied to the transformation of plant plastomes of any suitable taxon. Typically, the recipient plastome will be a plastome of a multicellular plant, usually a spermatophyte, which maybe a gymnosperm or an angiosperm. More typically the recipient plastome is an angiosperm plastome and is of a monocotyledonous or dicotyledonous plant, preferably a crop plant. Preferred dicotyledonous crop plants include tomato; potato; sugarbeet cassava; cruciferous crops, including oilseed rape; linseed; tobacco; spinach; sunflower; fibre crops such as cotton; horticultural crops such as gerbera and chrysanthemum; and leguminous crops such as peas, beans, especially soybean, and alfalfa. Tobacco is particularly preferred. Preferred monocotyledonous plants include graminaceous plants such as wheat, maize, rice, oats, barley, rye, sorghum, triticale and sugar cane. In general, preferred species will be ones that grow quickly and whose leaves form a major component of the biomass. As such, tobacco, horticultural crops and spinach are particularly preferred, particularly tobacco.

Stable Transplastomes

In transplastomic plants of the invention, the transplastomes will typically be stable transplastomes. The term stable, as used herein, refers to a transplastome in which internal recombination is not detectable over a period of time. Preferably, stability will be manifest by a lack of internal recombination within the transplastome after at least one cell division, for example, after up to ten cell divisions, or after up to one hundred cell divisions or more either in culture or during and/or after regeneration to give a first-generation plant. More preferably the stability is also retained in the second-generation plants that are progeny of the first-generation one and further progeny.

Methods of generating stable transplastomes are provided, in particular, in PCT/EP00/12446 (WO01/42441).

Thus, for example, a recipient plastome may be transformed with a transforming polynucleotide comprising:
(a) a 5′ sequence homologous to a region of the recipient plastome, and, joined thereto;
(b) a sequence heterologous to the recipient plastome comprising a xylanase-encoding region; and joined thereto;
(c) a 3′ sequence homologous to a region of the recipient plastome.

The transforming polynucleotide comprises homologous regions (a) and (c), which exist as flanking regions of the polynucleotide, that is, they define the 5′ and 3′ ends of the transforming polynucleotide. The homologous flanking regions allow insertion of the polynucleotide into the recipient plastome by homologous recombination.

The transforming polynucleotide further comprises a heterologous region (b) between the 5′ and 3′ homologous flanking regions (a) and (c). The heterologous region (b) does not posses substantial homology to any region of the plastome and, when integrated, therefore remains stable within the transplastome.

Xylanase-Encoding Sequences

Any suitable xylanase-encoding sequence may be used. According to the invention, a xylanase is a hydrolytic enzyme having the capacity to hydrolyse xylan. Xylanases can be classified into families F and G (now known as glycosidase families 10 and 11 respectively) on the basis of crystal structure. Xylanases from either of these families may be used according to the invention. Xylanases are “endo” acting enzymes and are also known a endo-1,4-xylanases (EC 3.2.1.8) are preferred. Xylanases have been detected in a number of microorganisms and microbial xylanases are preferred. For example, species of Bacillus, Streptomyces and Trichodema can all provide suitable xylanases. Thermostable xylanases are preferred. Alkali stable xyfanases are preferred. Particularly preferred is the xylanase encoded by the xynA of Bacillus sp. NG-27, as exemplified below. Some other examples are family G (11) xylanases of bacterial (e.g. Bacillus circulans) and fungal (e.g. Trichoderma harzianum) origin. In Trichoderma, two xylanases Xyn1 and Xyn2 are produced.

Regulatory Sequences

The xylanase-encoding polynucleotide will generally be under the control of a promoter. Any promoter capable of driving expression of the xylanase in the plant plastid concerned may be used. The promoter will typically be operably linked to the coding sequence, i.e. the promoter will be in such a position relative to the coding sequence that it can initiate transcriptions. Similarly, the coding sequence may be operably linked to a terminator (3′ untranslated region). Selectable or scorable marker sequences and other sequences may also be included in the transformation construct.

Prokaryotic and chloroplast promoters are preferred. More specifically, preferred promoters may be derived from the rice psbA gene promoter or the rice rrn gene promoter. Preferred terminators are derived from the 3′ untranslated region of the rice psbA gene or 3′ untranslated region of the rice rbcL gene. Preferred markers derived from the coding sequence of the aadA, uidA or NPTII genes. In the most preferred embodiment, the vector is pVSR 326 as exemplified below.

Cells for Transformation

The cell used for transformation may be from any suitable organism (see above list) and may be in any form. For example, it may be an isolated cell, e.g. a protoplast or single cell organism, or it may be part of a plant tissue, e.g. a callus, for example a solid or liquid callus culture, or a tissue excised from a plant, or it may be part of a whole plant. It may, for example, be part of an embryo, or a meristem, e.g. an apical meristem of a shoot. Preferably the cell is a cell containing chloroplasts, e.g. a leaf or stem cell, most preferably a leaf cell derived from the abaxial side of the leaf. Transformation may thus give rise to a chimeric tissue or plant in which some cells are transgenic and some are not.

Transformation Techniques

Generation of the transplastome is brought about by the insertion of the polynucleotide defined above. The polynucleotide may be inserted by any method known in the art, such as recombinant techniques, random insertion, or site directed integration. Preferably the method of polynucleotide insertion is site directed integration, more preferably by the process of homologous recombination. The transforming polynucleotide may be inserted into an isolated plastome or an in vivo plastome within a plastid. The plastid used may be in vivo or ex vivo. Insertion of the transforming polynucleotide is preferably performed by transformation of an in vivo plastid. Preferably, the plastid is within a cell, though it may be in isolated form.

Cell transformation may be achieved by any suitable transformation method, for example the transformation techniques described herein. Preferred transformation techniques include electroporation of plant protoplasts (Taylor and Walbot, 1985), PEG-based procedures (Golds et al, 1993), microinjection (Neuhas et al, 1987; Potrykus et al, 1985), injection by galinstrexpansion femtosyringe (Knoblauch et al, 1999) and particle bombardment (Boynton et al, 1988; Svab et al, 1990; Svab and Maliga 1993; U.S. Pat. No. 5,451,513; U.S. Pat. No. 5,545,817; U.S. Pat. No. 5,545,818; U.S. Pat. No. 5,576,198; U.S. Pat. No. 5,866,421). Particle bombardment is particularly preferred.

Selection of Transformed Cells and Generation of Homotransplastomic Cells

Homotransplastomic (see above) plastids, cells, plants, seeds, plant parts, plant tissues are preferred.

Cells generated by the transformation techniques discussed above will typically be present in chimeric tissues, and thus will be surrounded by other non-transformed cells. Furthermore, due to the multiple genome copies within each plastid, transplastomic plastids will typically contain multiple copies of untransformed plastomes. In order to produce homotransplastomic cells, that is, cells in which all plastids are homotransplastomic, in that all genomes within those plastids comprise the transforming polynucleotide of the invention, it is necessary to undergo rounds of screening. Screening will be carried out via an expressed selectable or scorable marker coding region, as defined above, in the integrated polynucleotide. Preferred selectable markers include the aadA, uidA and NPTII genes.

Homotransplastomic cells can be generated by multiple rounds of screening of the primary transformed cells for the presence of the selectable or scorable marker. Preferably, at least one round of screening is used, more preferably at least two rounds, most preferably three rounds or more. Typically the homotransplastomic nature of the thus generated cells are ascertained. Homotransplastomicity can be assayed by analysis of isolated plastomic DNA by Southern analysis or by performing polymerase chain reaction amplification. These techniques are suitably sensitive such that the presence of a single untransformed plastome could be detected.

Generating Stable Transplastomic Plants and Seeds

Transplastomic or homotransplastomic cells may be regenerated into a transgenic plant by techniques known in the art. These may involve the use of plant growth substances such as auxins, giberellins and/or cytokinins to stimulate the growth and/or division of the astomic or homotransplastomic cell. Similarly, techniques such as somatic embryogenesis and meristem culture may be used. Regeneration techniques are well known in the art and examples can be found in, e.g. U.S. Pat. No. 4,459,355, U.S. Pat. No. 4,536,475, U.S. Pat. No. 5,464,763, U.S. Pat. No. 5, 177,010, U.S. Pat. No. 5,187,073, EP 267,159, EP 604, 662, EP 672, 752, U.S. Pat. No. 4,945,050, U.S. Pat. No. 5,036,006, U.S. Pat. No. 5,100,792, U.S. Pat. No. 5,371,014, U.S. Pat. No. 5,478,744, U.S. Pat. No. 5,179,022, U.S. Pat. No. 5,565,346, U.S. Pat. No. 5,484,956, U.S. Pat. No. 5,508,468, U.S. Pat. No. 5,538,877, U.S. Pat. No. 5,554,798, U.S. Pat. No. 5,489,520, U.S. Pat. No. 5,510,318, U.S. Pat. No. 5,204,253, U.S. Pat. No. 5,405,765, EP 442,174, EP 486,233, EP 486,234, EP 539,563, EP 674,725, WO91/02071, WO 95/06128 and WO 97/32977.

In many such techniques, one step is the formation of a callus, i.e. a plant tissue comprising expanding and/or dividing cells. Such calli are a further aspect of the invention as are other types of plant cell cultures and plant parts. Thus, for example, the invention provides transplastomic or homotransplastomic plant tissues and parts, including embryos, meristems, seeds, shoots, roots, stems, leaves and flower parts. These may be chimeric in the sense that some of their cells are transplastomic or homotransplastomic and some are not. Similarly they may be chimeric in the sense that all cells are transplastomic but only some are homotransplastomic.

Regeneration procedures will typically involve the selection of transplastomic and/or homotransplastomic cells by means of marker genes, as discussed above. The regeneration step gives rise to a first generation transplastomic or homotransplastomic plant. The invention also provides methods of obtaining transplastomic or homotransplastomic plants of further generations from this first generation plant. These are known as progeny transplastomic or homotransplastomic plants. Progeny plants of second, third, four, fifth, sixth and further generations may be obtained from the first generation aspastomic or homotransplastomic plant by any means known in the art.

Thus, the invention provides a method of obtaining a transplantomic or homotransplastomic progeny plant comprising obtaining a second-generation transplastomic or homotransplastomic progeny plant from a first-generation transplastomic or homotransplastomic plant of the invention, and optionally obtaining transplastomic or homotransplastomic plants of one or more further generations from the second-generation progeny plant thus obtained.

Such progeny plants are desirable because the first generation plant may not have all the characteristics required for cultivation. For example, for the production of first generation transgenic plants, a plant of a taxon that is easy to transform and regenerate may be chosen. It may therefore be necessary to introduce further characteristics in one or more subsequent generations of progeny plants before a transplastomic or homotransplastomic plant more suitable for cultivation is produced. Progeny plants may be produced from their predecessors of earlier generations by any known technique. In particular, progeny plants may be produced by:
obtaining a transplastomic or homotransplastomic seed from a transplastomic or homotransplastomic plant of the invention belonging to a previous generation, then obtaining a transplastomic or homotransplastomic progeny plant of the invention belonging to a new generation by growing up the transplastomic or homotransplastomic seed; and/or
propagating clonally a transplastomic or homotransplastomic plant of the invention belonging to a previous generation to give a transplastomic or homotransplastomic progeny plant of the invention belonging to a new generation; and/or
crossing a first-generation transplastomic or homotransplastomic plant of the invention belonging to a previous generation with another compatible plant to give a transplastomic or homotransplastomic progeny plot of the invention belonging to a new generation; and optionally
obtaining transplastomic or homotransplastomic progeny plants of one or more further generations from the progeny plant thus obtained.

These techniques may be used in any combination. For example, clonal propagation and sexual propagation may be used at different points in a process that gives rise to a transplastomic or homotransplastomic plant suitable for cultivation. In particular, repetitive back-crossing with a plant taxon with agronomically desirable characteristics may be undertaken. Further steps of removing cells from a plant and regenerating new plants therefrom may also be carried out.

Also, further desirable characteristics may be introduced by transforming the cells, plant tissues, plants or seeds, at any suitable stage in the above process, to introduce desirable coding sequences other than the polynucleotides of the invention. This may be carried out by conventional breeding techniques, e.g. fertilizing a transplastomic or homotransplastomic plant of the invention with pollen from a plant with the desired additional characteristic. Alternatively, the characteristic can be added by further transformation of the plant obtained by the method of the invention, using the techniques described herein for further plastonic transformation, or by nuclear transformation using techniques well known in the art such as electroporation of plant protoplasts, transformation by Agrobacterium tumefaciens or particle bombardment. Particle bombardment is particularly preferred for nuclear transformation of monocot cells. Preferably, different transgenes are linked to different selectable of scorable markers to allow selection for both the presence of further transgenes. Selection, regeneration and breeding techniques for nuclear transformed plants are known in the art.

Obtaining Xylanase from Plants of the Invention

Plant Tissues and Plant Parts

Xylanase may be obtained, according to the invention, from any suitable plant tissue or part that contains transformed plastids of the invention. Generally, the transformed plastids of the invention will be chloroplasts so photosynthetic tissues such as stems and leaves are preferred. Leaves are particularly preferred, especially where the transplastomic plant of the invention is a tobacco plant.

Preferably, at least 1, at least 3, at least 5, at least 6, at least 8 or at least 10% of the total soluble protein expressed in the cells of the invention is recombinant xylanase according to the invention. Typically, the expression level will be around 5 or 6%, e.g. 3to 10%, 4 to 8% or 4 to 6%.

Preparing and Storing Tissues Prior to Extraction of Xylanase

The plant tissues of the invention may be harvested by conventional method. They may then also be dried or processed by any conventional methods. The Inventors have, surprisingly, found that 85% recovery of xylanase can be obtained in dried tobacco leaves. This is very important because it allows the farmer to retain dried leaves in his possession and sell them at a time convenient and profitable to him.

In the case of tobacco, the leaves may be sundried, or they may be artificially dried, e.g. at 30-35, 35-40, 40-42, 42-45, or 45-50° C. Drying at around 42° C. is preferred, e.g. at 40-44° C., especially at 42° C. Drying may be performed for any suitable period of time but drying over periods of days is preferred. For example, drying may take place over a period of 1 to 5 days, e.g. 2 to 4 days, for example 2, 3 or 4 days.

The Inventors have also found that, surprisingly, senescence of tobacco leaves do not impede good recovery of xylanase from the leaves. Therefore, the leaves may be allowed to scenesce before they are harvested.

Protein-Containing Extracts

From the transplastomic tissue of the invention, protein-containing extracts will typically be prepared. Such extracts may be made by any means known in the art, and will typically involve solubilisation of proteins contained in the tissue.

Heat Treatment

Heat treatment of the extract denatures at least some of the proteins from the extract, but the xylanase of the invention remains stable, and the extract is thus enlarged in xylanase. Accordingly, the xylanase of the invention is heat-stable to the conditions used according to the invention. By heat-stable is meant as that, for example, heat treatment causes no reduction in the activity of xylanase or causes only a small reduction, e.g. of 1, 2, 3-5 or 10%.

With this in mind, heat treatment may be carried out at any suitable temperature and over any suitable time. Temperatures in the region of 60° C. or 70° C. and times in region of 15 minutes to 1 hour are preferred. For example, heat treatment may be carried out at 50-55, 55-60, 60-65 or 65-70° C. or higher, depending on the heat-stability of the xylanase. Heat treatment at around 70° C., e.g. 65-75 or 68-72° C. is preferred, especially where enzyme used in the Examples are the xylanase of the invention. Heat treatment at any of the above-mentioned temperatures may be carried out for any suitable time, e.g. 15-20, 20-30, 30-40 or 40-60 minutes, depending on heat-stability of the xylanase. Heat treatment may be carried out before or after protein extraction.

It is preferred that the heat treatment step will lead to purification of the xylanase by a factor of 5 or more, or 10 or more.

Recovery of the Xylanase

Recovery of the xylanase of the invention may be carried out by any suitable means. Ammonium sulfate fractionation has been found to be simple and effective in the context of transplastomic tobacco, and is a preferred method. Where ammonium sulfate fractionation is used, it is preferred that, of the protein in the ammonium sulfate fraction, at least 80, at least 90 or at least 95% of the protein is xylanase protein of the invention.

It is preferred that the recovery stage results in a purification of 25 fold or more, 30 fold or more, or 35 fold or more.

It is preferred that at least 50, at least 70, at least 80, at least 85 or at least 90% recovery of activity is achieved. By this, we mean that at least 50, 70, 80, 85, or 90% is retained after recovery, compared to the activity retained when measurements are made on fresh tissue.

Uses of Xylanase of the Invention

Xylanases of the invention may be used in any context in industry for any activities that require xylanases. For example, they may be used in (1) the paper industry for the production of pulp with improved qualities, (2) baking, brewing and feed industry for the improvement of product quality, (3) conversion of xylan to monosaccharides that can be further converted into ethanol, (4) the preparation of complex polysaccharide diet for the monogastric animals and, (5) processing of plant fibers (e.g. flax and hemp) by selectively removing xylan components (Herbers et al, 1995, Liu et al, 1997).

In the paper industry, complete or partial digestion of xylan is an important alternative step to existing processes that make use of chlorine. Due to increased environmental awareness and to make the process more economical, there is a need to minimise the use of chlorine. A prior treatment of pulp with xylanase has been shown to decrease the consumption of chlorine and other bleaching agents in the subsequent stages, minimising the environmental impact of bleaching (Vikari et al, 1994). The xylanase produced and purified in the present study with thermostable and alkalistable properties should be highly desirable in paper industry as it can be used in the preparation of pulp without any major modification in the existing process. In addition, this enzyme should also find major application in bakery and food industry as it was 50% active at temperatures between 50-70° C. and the pH 6-11.

### EXAMPLES

Materials and Methods

Construction of Tobacco Plastid Expression Vector

The plastid transformation vector, pVSR326 (PCT/EP00/12446; WO 01/42441), was constructed using the rrn and psbA promoters and the 3′ untranslated regions of psbA and rbcL gene from rice plastome primary clones (Hiratsuka et al, 1988). The selectable aadA and reporter uidA genes were cloned from pUC-atpX-AAD (Goldschmidt-Clermont 1991) and pGUSN358-S (Clontech, Farrell and Beachy 1990) plasmids, respectively. The tobacco plastid genome sequences rbcL-accD genes (Shinozaki et al, 1986) were used for site specific integration of chimeric aadA and uidA genes into the plastid DNA.

The rice psbA gene promoter, psbARP, (nucleotides 1615-1141, EMBL Acc. No. X15901) was PCR amplified using pRB7 template DNA and SR01 (aaaactgcagtcgACTTTCACAGTTTCCATTCTGAA (SEQ ID NO: 1))—SR02 (catgcCATGGTAAGATCTTGGTTTATT (SEQ ID NO: 2)) primer combination. All subsequent PCR reactions were carried out in a 50 μl volume using 10 ng of DNA template, 0.2 mM dNTPs, 100 pmoles of each primer and the Pfu polymerase (Stratagene). The reaction was carried out for 25 cycles, each cycle being at 30 sec at 94° C., 30 sec at 50° C. and 2 min at 72° C. The resulting DNA was digested with restriction endonucleases SalI-NcoI and inserted upstream of the uidA gene in the plasmid pGUSN358-S to create pVSR100 intermediate vector. A multiple cloning site (MCS) was introduced into pVSR100 using SR03 (AATTGAGCTCGAGGTACCGCGGTCTAGAAGCTT (SEQ ID NO: 3))—SR04 (AATTAAGCTTCTAGACCGCGGTACCTCGAGCTC (SEQ ID NO: 4)) primers. The SR03 and SR04 primers are complementary to each other and provide cohesive ends that are compatible to EcoRI digested pVSR100 vector. The SR03 and SR04 oligos were designed in such a way the EcoRI site was not recreated upon ligation in the vector. The resulting plasmid was named pVSR200. The 3′ end of rice psbA gene, psbART, (nucleotides 81-134233 EMBL Acc. No. X15901) fragment was amplified using pRB7 template DNA and primers SR05 (attcgagctctaattaattaaGGCTITTCTGCTAACATATAG (SEQ. ID NO: 5)) and SR06 (ggggtacCATCATTTATTGGCAAA (SEQ ID NO: 6)). The amplified 3′ end of psbA gene fragment was digested with SacI-KpnI and cloned into pVSR200 to create pVSR300.

The 16S rRNA operon promoter, (16SRP) from rice (nucleotides 91, 100-91, 116, EMBL Acc. No. X15901) was PCR amplified using pRP7 template and primers SR07 (ctggggtacCTCCCCCCGCCACGATCG (SEQ ID NO: 7)) and SR08 (ggatcctcc tacactTCCAAGCGCTICAGATFATIAG (SEQ ID NO: 8)). The amplified DNA was digested with KpnI-BamHI and cloned into pBluescript II SK+(Stratagene) vector to create pBS16S. A 239 bp fragment of 3′ end of rbcL gene, rbcLRT, (nucleotides 55, 529-55, 784, EMBL Acc. No. X15901) was amplified using pRPI template DNA and SR09 AAGGTAGTTGGCAATAACTCGAGACTAAGTGGATAAAATTA (SEQ ID NO: 9)) and SR10 (gctctagaTTGTATTTATTTATTGTATTATAC (SEQ ID NO: 10)) primers. The first 18 bases in SR09 primer are complimentary to the 3′ end of the aadA gene and the last 18 bases are complimentary to 3′ end of the rbcL gene. A XhoI restriction site was introduced in between the aadA coding region and the 3′ end of rbcLRT, to facilitate easy exchange of aadA gene with any other selectable gene of interest. The amplified fragment, after gel purification, was used as the primer in the “Megaprimer” method of PCR (Sarkar and Sommer 1990) and SR11 (cgcggatccTATGGCTCGTGAAGCGGTTATC (SEQ ID NO: 11)) primer as the other primer and pUc-atpX-AAD as template DNA to amplify aadA coding region along with 3′ end of rbcL. The amplified product was digested with BaMHI-XbaI and cloned intopBS16S vector in the same sites to create p16SaadA vector. The aadA chimeric gene was taken as KpnI-XbaI fragment from p16SaadA and cloned into pVSR300 vector in the same sites to create pGUSaadAR vector.

The plastid targeting sequence from tobacco (nucleotides58, 056-60, 627; EMBL Acc. No. Z00044) was PCR amplified using SR12 (cccaagcttGAAAGAGATAAATTGAAC (SEQ ID NO: 12)) and SR13 (ccggaattcTATCTGAACTACTC (SEQ ID NO: 13)) primers and pTB22 (Shinozaki et al, 1988) as template DNA. The targeting sequences was digested with EcoRI-HindIII and cloned into pUC18 in the same restriction sites to create pUCFLK plasmid. A XhoI site present in the targeting sequence (nucleotide 60, 484; EMBL Acc. No. Z00044) was removed through site-directed mutagenesis in order to make XhoI site preset between aada coding region and 3′ end of rbcL as unique site in the vector pVSR326. Further, a ClaI site containing linker (GATCATCGAT (SEQ ID NO: 14)) was inserted into pUCFLK in between BamHI sites (nucleotides 59, 286 and 59, 306; EMBL Acc. No. Z00044) to create pUCFLKC. Plastid transformation vector, pVSR326, was created by introducing chimeric aadA and uidA containing sequences from pGUSaadAR as HindIII fragment at ClaI site of pUCFLKC after treating both the fragments with Klenow to generate blunt ends. Convenient restriction sites (underlined) with few extra bases were introduced into primers for easy cloning. Standard procedures were followed for PCR (Saiki et al, 1988) and cloning (Sambrook et al, 1989). The Pfu Polymerase (Stratagene) was used in all PCR reactions and promoter-junction regions were sequenced to detect any possible misincorporations during the PCR amplification.

The p326xynA was a derivative of vector pVSR326. The xynA coding region was PCR amplified from pGNG 19 (Gupta et al, 2000) using xly5 (GGAAGATCTTACCATGSTAAAAACGTTAAGAAAACC (SEQ ID NO: 15)) and xly3 (GGAAGTCTGAGCTCTATTAATCGATAATTCTCC (SEQ ID NO: 16)) primers and cloned at NcoI-SacI sites of pVSR326 by replacing uidA gene.

Plastid Transformation and Plan Regeneration

Tobacco (Nicotiana tabacum cv. Wisconsin 38) was transformed using particle delivery system PDS1000 (BioRad) according to the method described by (Svab and Maliga 1993). In brief, vector p326xynA DNA coated on to tungsten particles (M17 Bio-Rad) was bombarded on the in vitro grown tobacco leaf placed on RMOP medium (Svab and Maliga 1993), a modified MS medium (Murashige and Skoog 1962), containing 0.1 mg/l thiamine, 100 mg/l inositol, 3% sucrose, 1 mg/l BA and 0.1 mg/l NAA, 0.6% agar, pH 5.4). Transformed shoots were selected on RMOP medium containing 500 mg/l spectinomycin dibydrochloride. Three additional cycles of regeneration on esnomycin (500 mg/l) containing RMOP medium was carried out to obtain homotransplastomic plastid containing plants (Svab and Maliga 1993).

Nucleic Acid Analysis

Total DNA isolated from transgenic and control plants (Mettler 1987) were digested with relevant restriction endonucleases, separated on 0.8% agarose gels and transferred on to nylon membrane. About 3 μg of total RNA isolated from leaf tissue (Hughes and Galam 1988) was separated in denaturing formaldehyde agarose gel (1.5%) and blotted to nylon membranes. The membranes were UV crosslinked and then probed with ³²P labeled aadA, xynA and targeting rbcL-accD DNA fragments. Standard procedures were followed for hybridization (Sambrook et al, 1989) and membranes were subjected to autoradiography.

Zymography of Xylanase

Soluble leaf protein were extracted from the fresh/dried leaves either under Sun or at 42° C. from the greenhouse grown Nt. 3266xynA-1 and wild type plants in the extraction buffer (50 mM Tris-HCl pH 7.0, 5 mM DTT, 1 mM Na2EDTA, 0.1% SDS, 1% Triton X-100). Proteins were subjected to SDS-PAGE (Laemmli 1970). After the run, gel was washed extensively with 2.5% Triton X-100 in 0.05M Tris-Cl buffer pH 8.4 for 30 min followed by through rinsin 0.05M Tris-Cl buffer, pH 8.4. The gel was then laid on a xylan agar plate (2% agar, 1% xylan in 0.05M Tris-Cl, pH 8.4) and incubated at different temperatures for 2 h in a closed box with wet paper towels to keep the chamber moist. For the detection of xylanase in the leaf without extraction, leaves of the transgenic and wild type plants were excised, pressed against fine sand paper and placed on xylan agar gel followed by incubation at 70° C. for two hours. The xylan agar plate was then stained with 0.1% Congo red for 2 h and destained with 1M NaCl for several hours. Xylanase at was detected by the presence of yellow bands against the red background. For the identification of optimum pH requirement the gels were washed and incubated in buffers adjusted to various pH conditions. The gels were photographed after the staining.

Xylanase Assays

Substrate for xylanase was prepared as described before (Gupta et al, 2000). Briefly, 250 g of Oat spelts xylan (Sigma) was suspended in 250 ml of 0.05M Tris-Cl buffer (pH 8.4). Xylan suspension was sonicated for 30 minutes and the suspension was autoclaved at 15 psi for 20 minutes and brought to room temperature. Suspension was centrifuged at 16270 g for 30 minutes and the supernatant, which contained 8.5 mg ml-1 xylan, was used as the substrate for enzyme estimation Xylanase activity was measured in terms of amount of reducing sugars released from Oat spelts xylan by the enzyme following the method described by Miller (1959). The hydrolysis products of xylan by xylanase were analyzed by paper chromatography. Leaf extract from Nt. 326xynA-1 plant was incubated with 1% oat spelt xylan solution (pH 8.5) in a total volume of 1 ml at 50° C. After 48 hours, 50 μl of reaction mix was spotted on a Whatman 3 mm paper. For a direct comparison, highly purified maltose, xylose, and xylobiose obtained from Sigma were also included in the chromatography. As a positive control, an E. coli expressed and purified xylanase was also included in the chromatography. The chromatogram was developed according to the method described by Travelyn et al (1950).

Xylanase Purification

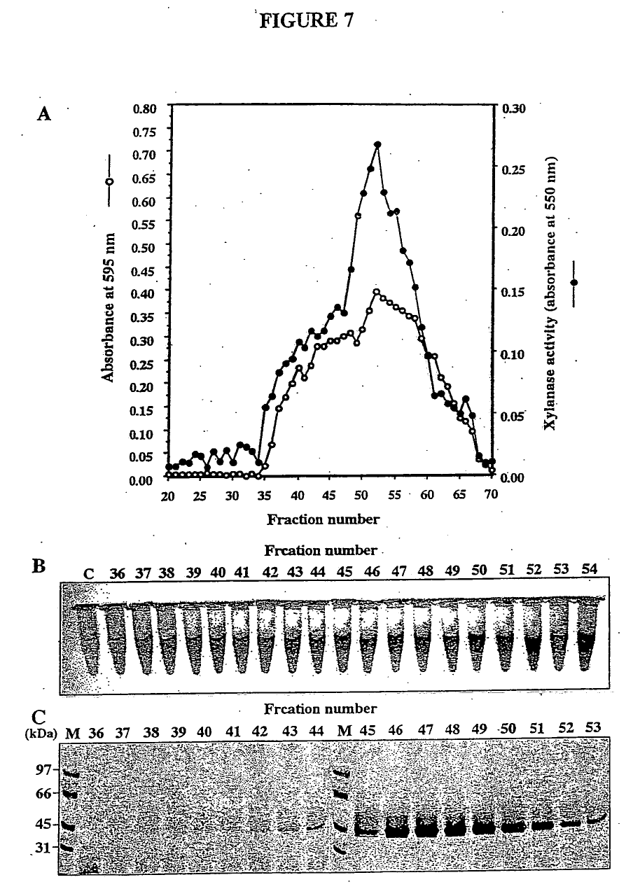

Soluble leaf protein were extracted from the fresh/dried leaves either under Sun or at 42° C. from the greenhouse grown Nt 3266xynA-1 progeny plants in a buffer containing 50 mM Tris pH. 8.3 and protease inhibitors (Complete tablets from Roche Biochemicals was used). The extract was passed through 4 layers of cheese cloth and heated to 70° C. The extact was centrifuged at 10,000 g and the clarified extract was loaded on to Q-sepharose column Etat was equilibrated with 50 mM Tris pH 8.3. The column was washed Massiv and the bound proteins were eluted using the a salt gradient (NaCl 0 M to 1.0 M concentration). Fractions were assayed for the xylansase activity (FIG. 7) and the active fractions were checked on the SDS-PAGE gels for purity.

Results

Expression Vector for xynA in Tobacco Chloroplasts

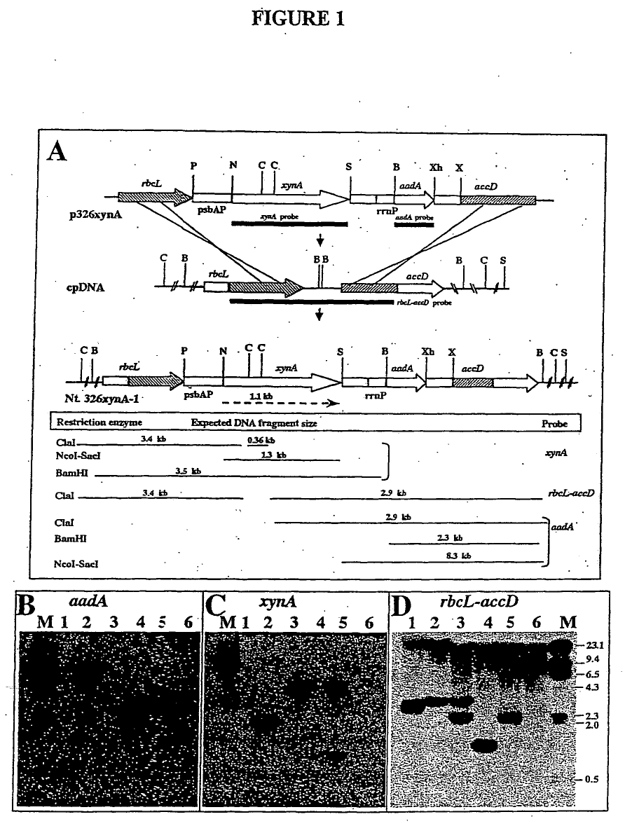

For high level expression of XynA in tobacco plants, a chloroplast transformation vector p326xynA was constructed (FIG. 1A). The p326xynA is a derivative of vector pVSR326 that contained a selectable aadA gene that confers resistance to spectinomycin/streptomycin and a reporter uidA gene. The aadA and uidA genes were put under the regulation of rice rrn and psbA gene promoters, respectively. The vector p326xynA was obtained by exchanging the coding region of uidA with that of xynA. The rbcL-accD gene sequences derived from tobacco plastid genome were provided in the vector flanking the chimeric xynA and aadA genes for site-specific integration through two homologous recombinations. The direction and the expected size of transcripts of xynA and aadA genes, a possible mechanism for transgene integration into tobacco plastome and the size of DNA fragments from restriction digestion with relevant enzymes when integrated into plastid genome were depicted in FIG. 1A.

Transformation and Regeneration of Stable Transplastomic Plants

The particle bombardment of leaf tissue with DNA of vector p326xynA was followed for chloroplast transformation under spectinomycin selection. Although the vector DNA is randomly delivered into ink leaf cells in particle bombardment method, the selectable aadA gene is expected to express and confer resistance to spectinomycin only when it enters the chloroplasts because of the high specificity of the rrn promoter in chloroplasts. Homotransplastomic lines were established by repeating regeneration process three times from the leaf issues of primary transformants under spectinomycin selection. Out of 26 green shoots regenerated on spectinomycin selection from 20 bombardments, 16 plants were found to be positive for the presence of xynA and aadA genes.

Stable Integration of xynA and aadA into Plastid Genome

Southern hybridization analysis using xynA, aadA and rbcL accD probes confirmed the stable integration of vector DNA into tobacco plastid genome (FIG. 1B). The total genomic DNA isolated from Nt. 326 xynA-1 and wild type plants were digested with ClaI, BamHI, and NcoI-SacI restriction enzymes and then probed with xynA, aadA and rbcL-accD gene probes. As shown in FIG. 1B-D, the sizes of the DNA fragments hybridized to the xynA and aadA in Nt 326xynA-1 plant, were in agreement with the expected size of DNA fragments with transgenes integrated in the plastid genome site-specifically. Presence of the 3.4 kb fragment in the wild-type plant (FIG. 1B, lane 5) and 3.4 kb and 2.9 kb fragments in Nt. 326xynA-1 plant (FIG. 1B, lane 6) when probed with the targeting rbcL-accD sequences confirmed the site-specific integration of xynA and aadA into plastid genome specified by the targeting sequences. The complete absence of 3.4 kb signal in Nt 326xynA-1 plant was a clear evidence for the homoplasmic nature of the transplastome. Hybridization of targeting sequences with the NcoI-SacI (lanes 1 and 2) and BamHI (lanes 3 and 4) digested DNA further confirmed the site-specific integration of xynA and aadA sequences. Direct evidence for the stable integration of xynA and aadA sequences into plastid DNA was obtained by reprobing the same blot with the coding regions of xynA (FIG. 1C) and aadA (FIG. 1D) respectively. When the same blot was hybridized with xynA probe, DNA fragments of 1.3 kb, 3.5 kb and 3.4 kb & 360 bp size were observed only in the lanes containing genomic DNA obtained from the transformed plant and digested with the restriction enzymes NcoI & SacI, BamHI and ClaI, respectively. Similarly, when the blot was hybridized with aadA probe, DNA fragments of 8.3 kb, 2.3 kb and 29 kb size were observed only in lanes containing genomic DNA from transformed plants and digested with restriction enzymes NcoI & SacI, BamHI and ClaI, respectively. No signal was observed in the wild type plant DNA containing lanes, probed with xynA or aadA coding sequences.

Expression of Chimeric xynA

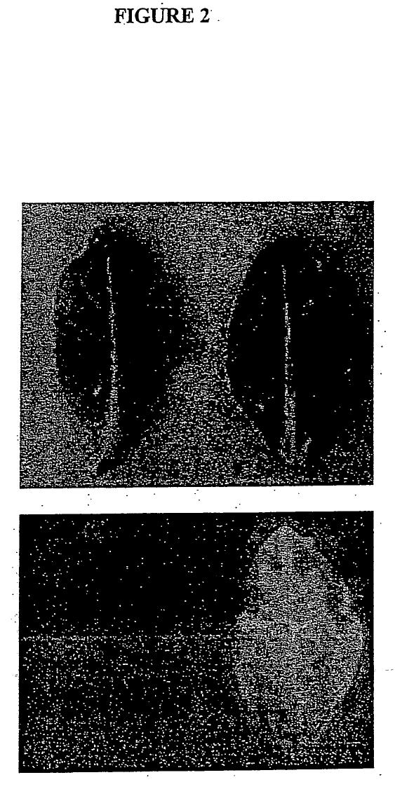

Northern blot analysis was performed to confirm the wanton of chimeric xynA gene and the results are presented in FIG. 1E. As can be seen in FIG. 1E, a 1.3 kb transcript corresponding to the expected size of xynA was observed in the RNA isolated from Nt. 326xynA-1 plant when probed with the coding region of xynA. Reprobing the same blot with aadA probe revealed the presence of a ˜1.0 kb transcript corresponding to the expected size of aadA mRNA. Zymography showed the presence of enzymatically active xylanase in the transplastomic plant leaves. As can be seen from FIG. 2A, the activity of the xylanase was present in the transformed plant leaves whereas no activity could be detected in the untransformed wild type plant leaf. It could be noted that the activity was found to be uniform and present all along the leaf. In order to verify the molecular size of the expressed xylanase in the tobacco chloroplasts, total protein were extracted from the transformed and wild type plants, separated on an SDS-PAGE gel and subjected to zymography to detect the xylanase activity. A single band corresponding to the molecular size of 42 kDa was observed in the transformed plant leaf extract (data not shown) confirming the expression of active xylanase in the transplastomic plants.

Purification of Xylanase

Purification steps followed for xylanase from the greenhouse grown plant leaves are presented in Table 1.

TABLE 1
Purification of xylanase from the greenhouse grown plant leaves

| | Total | Total | | |
|---|---|---|---|---|
| | protein | activity | Specific | Yield |
| Purification step | (mg) | (units) | activity¹ | (%) |
| From the fresh leaves* | | | | |
| Crude extract | 377.0 | 14075 | 37 | 100.0 |
| Heat treatment | 33.9 | 10000 | 295 | 71.3 |
| Ammonium sulphate | 10.9 | 12000 | 1100 | 85.5 |
| Precipitation (50-75%) | | | | |
| From the leaves | | | | |
| dried at 42° C. # | | | | |
| Crude extract | 95.0 | 4263 | 45 | 100.0 |
| Heat treatment | 8.5 | 3464 | 406 | 81.3 |
| Aminonium sulphate | 2.8 | 3097 | 1106 | 72.7 |
| Precipitation (50-75%) | | | | |

¹nmoles of xylose produced/min/mg protein.
Starting from 100 grams (*) and 25 grams (#) of fresh leaves.

Starting from 100 grams (*) and 25 grams (#) of fresh leaves.

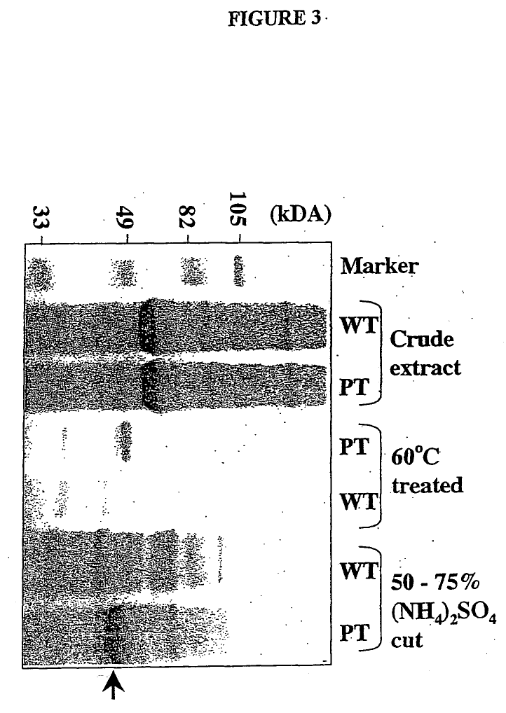

Incubation of leaf extracts at 60° C. for 30 minutes resulted in a 11 fold purification. Ammonium sulfate fractionation resulted in a 35 fold purification with 85% recovery of activity. Total proteins extracted from wild type and transplastomic plants, purified through various steps, were analyzed on SDS-PAGE gels. A distinct band of 42 kDa, which corresponded to the bacterially expressed enzyme (data not shown), was observed in the transplastomic plant leaf extracts (FIG. 3). The relative amount of xylanase was calculated to be ˜6% of the total protein as determined densitometrically using Kodak ID Image analysis software. The ammonium sulfate fraction (50%-75%) contained a single major protein (95%) that corresponded with the zone of xylanse activity it the zymogram. Alternately, the xylanase was purified further using sepharose (Pharma) chromatography.

Soluble leaf protein were extracted from the fresh/dried leaves either under Sun or at 42° C. from the greenhouse grown Nt 3266xynA-1 progeny plants, passed through 4 layers of cheese cloth and heated to 70° C. The clarified extract was loaded on to Q-sepharose column equilibrated previously with 50 mM Tris pH 8.3 The column was washed extensively and the bound proteins were eluted using the a salt gradient. Fractions containing xylanase were identified using xylansase activity assay (FIG. 7). The active fractions when were checked on the SDS-PAGE gels showed a single band corresponding to the size of xylanase (FIG. 7). Xylanase eluted between 100 mm to 300 mM NaCl concentration. The total xylanase activity based on the fresh leaf weight was estimated to be 140755 U per kg.

Activity of Xylanase in the Leaves Dried under Sun for 2-4 days or at 42° C. Or 48 hrs and in the Leaves Undergoing Senescence

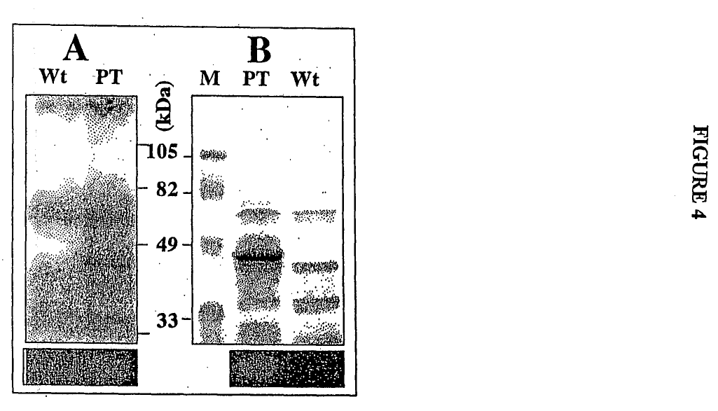

Total proteins extracted from the dried leaves were heated to 60oC for 30 minutes and precipitated with ammonium sulfate (50-75%) and tested for the presence of xylanase activity. In a Coomassie blue stained gel, a distinct 42 kDa protein was observed in the transformed plant which corresponded with the zone of activity of the xylanase in the zymogram (FIG. 4A). The total activity of xylanase in the dried leaves starting from 25 grams of fresh leaves was found to be 4263 U (Table 1). Similar experiments involving the leaves that were undergoing senescence revealed the presence of enzymatically active xylanase (FIG. 4B) with an estimated total activity of 4995 U in 25 grams (fresh weight) of leaves.

Temperature and pH Requirement for the Optimal Activity of Chloroplast Expressed Xylanase

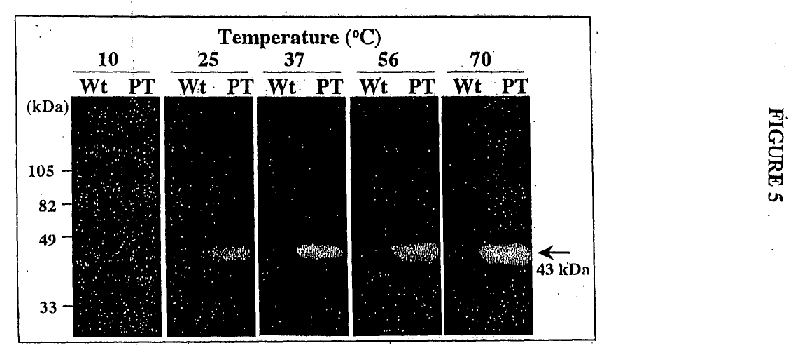

Characterization of xylanase activity using oat spelts xylan indicated that the chloroplast expressed enzyme is biologically active at a pH range of 6-11 with the peak activity at pH 8.4. The enzyme was less than 50% active at pH 5.6, the physiological pH of the plant cells. The recombit xylanase was active between 25° C.-85° C. with the optimum activity at 70° C. (FIG. 5). The enzyme activity was less than 25% at 35° C. and below.

Chloroplast Expressed Xylanase Retains its Substrate Specificity

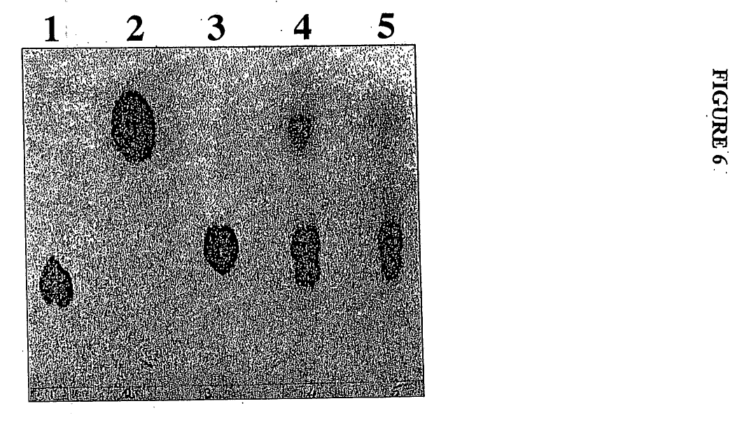

The specificity of substrate for chloroplast expressed enzyme was determined using oat spelt xylan. Up on paper chromatography, it showed that the major hydrolysis products of xylan were xylobiose and zylose, identical to the specificity observed for the E. coli expressed enzyme (FIG. 6).

Analysis of T1 Generation for Growth and Yield Parameters

All spectinomycin resistant T0 plants were transferred to the greenhouse to allow them to flower and set seeds. Reciprocal hybridizations were carried out to test the maternal inheritance of the aadA and xynA genes among the progeny. The T1 generation plants obtained from the self pollination of T0 plants were grown to maturity in the greenhouse conditions and various critical growth associated parameters and the yield of plants were recorded. All transplastomic plants appeared similar in both morphology and fruiting to non-transgenic tobacco plants raised under same growth conditions. There was no significant difference between the transplastomic and wild type plants for plant height, flowering time and leaf size indicating lack of any adverse affect on the growth of the plants due to high level expression of XynA in chloroplasts. Similarly, high levels of xynA expression in the chloroplasts did not affect the chlorophyll content of the transplastomic plants. Further, there were no changes in yield related parameters such as number of pods per plant and the weight of the pod in the formed plants when compared to wild type plants under the greenhouse conditions.

### REFERENCES

Bendich, A. J. (1987) BioEssays 6, 279-282.

Borsjuk, N. V., Borisjuk, L. G., Logendra, S., Petersen, F., Gleba, Y & Raskin, I. (1999) Nature Biotechnology 17: 466-469.

Goldschmiidt-Clermont, M. (1991) Nucl. Acids Res. 19, 4083-4089.

Gupta N., Maiti, S., Reddy, V. S. & Ghosh, A. (2000): Appl. Environ. Microbiol. 66: 2631-2635.

Herbers, K., Wilke, I & Sonnewald U. (1995) Nature Biotechnology 13: 63-66.

Hiratsuka J., Shimada, H., Whittier, R. Ishibashi, T., Sakamoto, M., Mori, M., Kondo, C., Honji Y., Shun, C. -R., Meng, B. -Y., Li, Y. -Q., Kanno, A., Nishizawa, Y., Hirai, A., Shinozaki, K. & Sugiura, M. (1988) Mol. Gen. Genet. 217, 185-194.

Hughes, D. & Galam, G. (1988) Plant Mol. Biol. Rep. 6, 253-257.

Kota, M., Daniell, H., Varma, S., Garczynski, S. F., Gould, F. & Moar, W. J. (1999) Proc Natl Acad Sci U S A. 96, 1840-1845.

Laemmli, U. K (1970) Nature 227, 680-685.

Liu, J. H., Selinger, L. B., Cheng, K. J., Beauchemin K. A & Moloney, M. M. (1997) Mol. Breed. 3: 463-470.

Mettler, I. J. (1987) Plant Mol. Biol. Rep. 5, 346-349.

Miller, G. L. (1959) Anl. Chem. 31: 426-428.

Murashige, T. & Skoog, F. A. (1962) Physiol. Plant. 15, 473-497.

Saiki, R. K., Gelfand, D. H., Stofell, S., Scharf, S. J., Higuchi, R., Horn, G. T., Mullis, K. B. & Erlich, H. A. (1988) Science 239, 487-491.

Sambrook, J., Fritsch, E. F. and Maniatis, J. (1989) Molecular cloning. A Laboratory Manual (Cold Spring Harbor Laboratory Press, Cold Spring Harbor, N.Y).

Sarkar, G. & Sommer, S. S. (1990) Biotechniques 8, 404-407.

Shimozaki, K., Ohme, U, Tanaka, M., Wakarugi, T., Hayash-ida, H., Matsubayashi T., Zaita, N., Chunwongse, J., Obokata, J., Yamaguchi-Shinozaki, K., Ohito, C., Torazawa, K, Meng, B. Y., Sungita, M., Deno, H., Kamogahsira, T., Yamada, H., Suguira, M. (1986) EMBO J. 5, 2043-2049.

Staub, J. M., Garcia, B., Graves, J., Hajdukiewiez, P. T., Hunter, P., Nehra, N., Paradkar, V., Sohilttier, M., Carroli, J. A. Spatoia, L., Ward, D., Ye, G & Russell D. A (2000) Nature Biotechnology 18: 332-336.

Svab, Z. & Maliga, P. (1993) Proc. Natl. Acad. Sci. 90, 913-917.

Trevelyn, W. E., Procter, D. P. & Saddler, J. H. (1950) Nature 166: 444-445.

Vikari, L., Kantetinen, A., Sundquist, J. & Linko, M. (1994) FEMS Microbiol. Rev. 13: 335-350.

Wong, K. K. Y., Tan, L. U. L & Saddler, J. H. (1988) Microbiol. Rev. 52: 305-317.

1. A process of obtaining a xylanase, said process comprising:
providing a protein-containing extract of a transplastomic plant tissue comprising plastids transformed with a polynucleotide encoding said xylanase, said extract having been subjected to heat treatment that has denatured at least some of the protein content of said tissue but under which the xylanase has remained stable; and
recovering said xylanase from said extract.

2. A process of obtaining a xylanase, said process comprising:
providing a transplastomic plant tissue comprising plastids transformed with a polynucleotide encoding said xylanase;
preparing a protein-containing extract therefrom;
subjecting said extract to heat treatment that denatures at least some of the protein content of said extract but under which the xylanase remains stable; and
recovering said xylanase.

3. A process of obtaining a xylanase, said process comprising:
transforming a plant cell with a polynucleotide encoding said xylanase, thereby to obtain a transplastomic cell comprising plastids transformed with a polynucleotide encoding said xylanase;
regenerating a transplastomic plant from said transplastomic cell;
providing a transplastomic plant tissue from said plant;
preparing a protein-containing extract therefrom;
subjecting said extract to heat treatment that denatures at least some of the protein content of said extract but under which the xylanase remains stable; and
recovering said xylanase.

4. A process according to claim 1 wherein said plastids are chloroplasts and/or wherein said plant tissue is homotransplastomic.

5. A process according to claim 1 wherein recovery of said xylanase comprises ammonium sulfate fractionation, and optionally one or more further purification steps.

6. A process according to claim 1 wherein said heat-treatment is at a temperature of 60° C. or above.

7. A process according to claim 1 wherein the transplastomic plant tissue has undergone senescence and/or has been sun-dried or artificially dried, optionally at a temperature of 42° C. or above.

8. A process according to claim 1 wherein said xylanase is a bacterial or fungal xylanase.

9. A process according to claim 8 wherein said xylanase is encoded by the xynA gene of Bacillus sp NG-27.

10. A process according to claim 1 wherein said plant tissue is tobacco plant tissue.

11. A process according to claim 10 wherein said plant tissue is tobacco leaf tissue.

12. A process according to claim 1 wherein the polynucleotide encoding the xylanase is operably linked to a prokaryotic or chloroplast promoter.

13. A process according to claim 1 wherein the polynucleotide encoding the xylanase is operably linked to a rice rrn or psbA promoter and/or to a psbA or rbcl 3′untranslated region.

14. A process according to claim 1 wherein:
the xylanase accounts for 5% or more of the total tissue protein; and/or
where ammonium sulfate fractionation is used, the ammonium sulfate fraction 90% or more of the protein in the ammonium sulfate fraction is xylanase;
from tissue as defined in claim 7, recovery of 50% or greater or 80% or greater, of the xylanase activity present is obtained.

15. A transplastome transformed with a polynucleotide encoding a xylanase, optionally a xylanase as defined in claim 8.

16. A transplastomic or homotransplastomic plastid comprising a transplastome as defined in claim 15.

17. A plastid according to claim 16 which is a chloroplast.

18. A transplastomic or homotransplastomic cell comprising a plastid as defined in claim 16, or a transplastomic or homotransplastomic plant, plant seed, or plant tissue comprising said cell.

19. A plant, plant seed, or plant tissue according to claim 18 wherein the xylanase is one which remains stable under conditions that denature at least some of the protein content of said plant, seed or tissue but under which the xylanase.

20. A plant, plant seed, or plant tissue comprising the cell of claim 18.

21. A process of obtaining a xylanase comprising expressing said xylanase in a cell, plant, seed or tissue as defined in claim 18 and recovering said xylanase therefrom.

22. A process according to claim 1 further comprising employing the xylanase obtained in the manufacture of paper, for improvement of product quality in baked or brewed products or feed; in the conversion of xylan to polysaccharides, optionally for further conversion to ethanol; in the preparation of complex polysaccharide diets for monogastric animals; or in the processing of plant fibres by selective removal of xylan components.

23. A xylanase obtained by the process of claim 1.

24. A method comprising using the xylanase obtained by the process of claim 1 in the manufacture of paper; for improvement of product quality in baked or brewed products or feed; in the conversion of xylan to polysaccharides, optionally for further conversion to ethanol; in the preparation of complex polysaccharide diets for monogastric animals; or in the processing of plant fibres by selective removal of xylan components.
